(19) United States
(12) Patent Application Publication
(10) Pub. No.: US 2021/0087507 A1
(43) Pub. Date: Mar. 25, 2021

(54) GRAVITY DRIVEN BIOREACTORS AND METHODS OF OPERATION

(71) Applicant: ExxonMobil Research and Engineering Company, Annandale, NJ (US)

(72) Inventors: Everett J. O'Neal, Asbury, NJ (US); Yesim Igci, Mountainside, NJ (US); Patrick L. Hanks, Bridgewater, NJ (US); Randall J. Lamutt, Aurora, CO (US); Mark A. Deimund, Jersey City, NJ (US); Vinit Choudhary, Cypress, TX (US)

(21) Appl. No.: 17/019,405

(22) Filed: Sep. 14, 2020

Related U.S. Application Data
(60) Provisional application No. 62/904,039, filed on Sep. 23, 2019.

(51) Int. Cl. C12M 1/00; A01G 33/00; B01D 61/14

(52) U.S. Cl. CPC C12M 21/12; B01D 61/142; B01D 61/145; A01G 33/00

(57) ABSTRACT

A system for growing algae in a slurry, includes a pond having an inlet and an outlet and a plurality of contiguous elongate channel segments coupled fluidically in sequence between the inlet and the outlet, and the inlet is located at an elevation higher than outlet to allow gravity to flow the slurry from the inlet to the outlet. The system further includes a separation device fluidically coupled to the outlet to receive and separate algae from the slurry.

### CROSS-REFERENCE TO RELATED APPLICATIONS

This application claims the benefit of priority from U.S. Provisional Application No. 62/904,039 filed Sep. 23, 2019, which is herein incorporated by reference in its entirety.

### BACKGROUND OF THE INVENTION

Concerns about climate change, carbon dioxide (CO₂) emissions, and depletion of subterranean oil and gas resources have led to widespread interest in the production of biofuels from algae and microalgae. As used herein, the term “biofuel” refers to any type of fuel produced from algae, and the term “algae” will include microalgae, unless explicitly distinguished.

As compared to some other plant-based biofuel feedstocks, algae have higher CO₂ fixation efficiencies and growth rates, and growing algae can efficiently utilize wastewater and industrial gases as nutrient sources. The biomass of algae stores increasing quantities of lipids as it grows. Methods for harvesting and utilizing algae involve extracting and converting their stored lipids and carbohydrates into renewable biofuels, such as diesel and jet fuel, or into other hydrocarbons, as examples.

Algae biomass is generally grown in a water slurry contained in a bioreactor system. Algae bioreactors are sometimes referred to as “photobioreactors” since they utilize a light source to cultivate algae, which are photoautotrophic organisms, or organisms that can survive, grow, and reproduce with energy derived entirely from the sun through the process of photosynthesis. Photosynthesis, aided by other cellular biochemical processes, is essentially a carbon recycling process through which inorganic CO₂ is absorbed and combined with solar energy, nutrients, and water to synthesize carbohydrates, lipids, and other compounds necessary to algae life. In addition to production of lipids and carbohydrates for biofuel production, the benefits of growing and harvesting algae includes utilization of CO₂ and production of oxygen.

The most common types of bioreactors used in algal cultivation are open channel ponds and tubular-type enclosed or open reactors. One goal for open channel pond bioreactors is to integrate and interact with the surrounding environment in a manner that reduces environmental impact, capital expenses, and operating costs.

### SUMMARY OF THE INVENTION

The present disclosure is related to biofuel production from algae and, more particularly, to gravity-driven bioreactor pond systems that include long troughs designed to continuously flow and grow algae.

In some embodiments disclosed herein, a system for growing algae in a slurry, includes a pond having an inlet and an outlet and a plurality of contiguous elongate channel segments coupled fluidically in sequence between the inlet and the outlet, and the inlet is located at an elevation higher than outlet to allow gravity to flow the slurry from the inlet to the outlet. The system further includes a separation device fluidically coupled to the outlet to receive and separate algae from the slurry.

In some embodiments disclosed herein, a method for growing algae includes containing a slurry that includes algae in a pond having an inlet and an outlet and a plurality of contiguous elongate channel segments coupled fluidically in sequence between the inlet and the outlet, wherein the inlet is located at an elevation higher than outlet, flowing the slurry between the inlet and the outlet under force of gravity, receiving the slurry from the pond at a separation device, and separating the slurry into algae and a remainder of the slurry with the separation device.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a schematic diagram of an example system for growing algae, according to various embodiments of the present disclosure.

FIG. 2 is a schematic diagram of another example system for growing algae, according to various embodiments of the present disclosure.

FIG. 3 is a schematic diagram of another example system for growing algae, which may incorporate multiple of the systems of FIG. 1 or FIG. 2, according to various embodiments of the present disclosure.

### DETAILED DESCRIPTION

The present disclosure is related to biofuel production from algae and, more particularly, to gravity-driven bioreactor pond systems that include long contiguous troughs designed to continuously flow and grow algae.

Algae ponds and pond systems that include an elongate, serpentine trough are disclosed herein. The flow of algae through the ponds is gravity-driven and, in at least some modes of operation, algae can fully develop and mature while flowing only once through the serpentine pond system from inlet to outlet. A pond or system configured for once-through performance with respect to the travel of algae may alternatively be described as a single-pass pond or system. The pond systems described herein may include recycling of water and nutrients from an outlet to an inlet of a pond. Because the flow of the algae through the ponds is generally gravity-driven, paddlewheels and other sources of active, mechanical mixing or agitation may not be required.

Various embodiments disclosed herein provide one or more techniques (e.g., configurations or methods) to compensate for varying algae growth rates and algae concentrations along the fixed length of the ponds. A first technique is the use of membrane filter units and recirculation pumps for permeate, which provide a harvesting method that is less dependent on the concentration of algae than other harvesting methods. A second technique is to vary the concentration or quantity of algae seed material fed to the ponds disclosed herein. A third technique is to build a system having multiple ponds that can be operated in parallel or can be operated as groups of two, three, four, or more ponds operating in series to increase residence time within the groups of ponds. The second and third techniques may reduce the variation of algae received by a device that skims, filters, or otherwise harvests the algae.

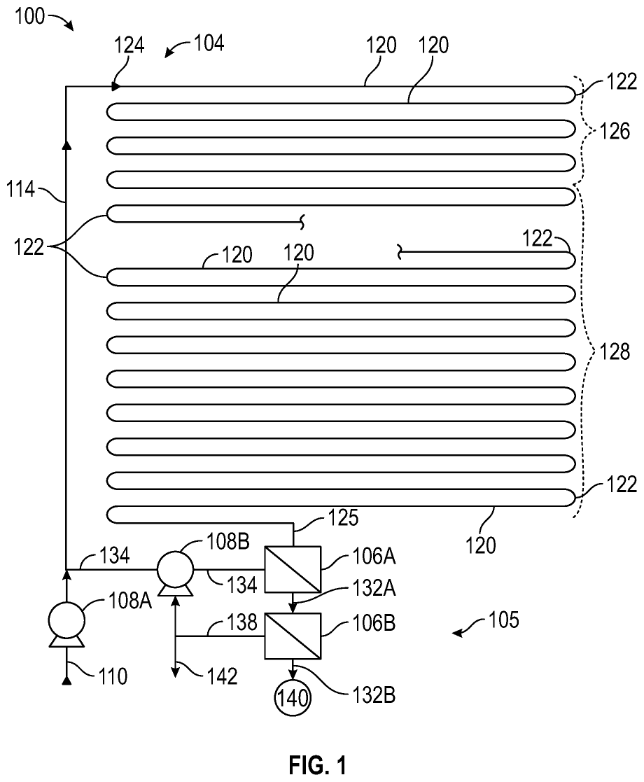

FIG. 1 is a schematic diagram of an example system 100 for growing algae in an algae slurry, according to one or more embodiments. As used herein, the term “algae slurry,” and grammatical variants thereof, refers to a flowable liquid comprising at least water, algae cells, and algae nutrient media, discussed in further detail herein, below. As illustrated, system 100 includes a pond 104 formed or shaped as an elongate, serpentine trough, and includes a filtration and pumping system 105. In some embodiments, the depth of the algae slurry within pond 104 may be range between about 5 inches (in.) and about 12 in. to facilitate sufficient sunlight penetration needed for proper algae growth. In other embodiments, however, the depth of the algae slurry may be less than 5 in. or greater than 12 in., without departing from the scope of the disclosure. The algae slurry may be gravity-driven as it flows in the pond 104 and the algae grown in and harvested from pond 104 may pass through pond 104 once before being harvested. Accordingly, an at least some embodiments, system 100 may perform as a once-through, gravity-driven system. Though not shown, an algae seed feed system may be included in the system 100 to provide a continuous or periodic input of algae for growth, which will facilitate a constant algae stream at the exit to pond 104.

As illustrated, pond 104 is in the general shape of an elongate, serpentine trough or channel. By being constructed on a sloping or graded region of land, the algae slurry contained within pond 104 may flow along the trough or channel under the force of gravity. Pond 104 may be exposed to the sunlight, or may alternatively employ artificial light to help facilitate growth of the algae. Pond 104 has an inlet 124 and an outlet 125, and is defined by a plurality of elongate channel segments 120 coupled in series by a plurality of channel bends 122 and extending between the inlet 124 and the outlet 125, and thus forming the elongate, serpentine flow path for algae slurry. The sequentially coupled channel segments 120 may be arranged substantially parallel to each other or, alternatively, may be “wrapped around” a hill, as examples. In theory, there is no limit to the number of channel segments 120 and bends 122 that may be included in pond 104. In various embodiments, pond 104 may encompass a land region within the range of 200 to 5,000 acres (0.81 to 20.2 km²); although, some embodiments may include a larger pond 104 and others may include a smaller pond 104.

Inlet 124 is located at an elevation that is higher than outlet 125. For example, in some embodiments, inlet 124 is located at an elevation within the range 24 to 37 meters (“m”) (80 to 120 feet (“ft”)) above outlet 125, and may be, for example, 30 m (100 ft) above outlet 125. Other greater or lesser elevation differences are possible for pond 104. In various embodiments, channel segments 120 and channel bends 122 may be carved or dug into the land region or may be built on top of the land region. Pond 104 may be built to follow an existing grade or slope of a land region on which pond 104 is built. One or more of the channel segments 120 may be sloped downward with respect to inlet 124. One or more of the channel bends 122 may be sloped downward with respect to inlet 124.

The existing slope or a constructed slope of a land region on which pond 104 is built may influence a selection of length for channel segments 120 to achieve a desired flow velocity, residence time, or another parameter. For example, a land region having a relatively steeper slope may be better suited for a shorter channel length than a land region having a shallower slope. If the slope of a land region on which pond 140 is built varies, the length of various channel segments 120 may be adjusted to compensate. When constructing pond 104 on land having an existing slope that is greater than the planned construction slope, a central portion of the pond may be built on the existing land with minimal excavation, an upper portion of the pond may be built using greater excavation of the land, and a lower portion of the pond may be built with the addition of fill-soil or aggregate, which may come from the region of the upper portion of the pond. Some embodiments include a grade or slope in the range of 0.5% to 1% for at least a portion of pond 104. Other values of slope less than 0.5% or greater than 1% of may be feasible.

The growth of algae in pond 104 may include a growth phase and a lipid phase. During the lipid phase, algae grows or amasses increasing quantities of lipids or oil substance, which may be particularly beneficial when harvesting and processing the algae to produce biofuels. Pond 104 may include a first pond section 126 in which algae may proceed through the growth phase and a second pond section 128 in which algae may proceed through the lipid phase. The division between pond sections 126, 128 may be a result of design decisions, may be a result of operating conditions (e.g., flow rate of water supply, speed of water in the pond, nutrients, available sunlight, etc.), or may be attributable to a combination of these factors. The division between pond sections 126, 128 may indicate a location along pond 104 where a majority of the algae flowing along pond 104 has reached a particular or a general level of maturity and has begun to produce lipids at an increased rate. The location where pond section 126 ends and pond section 128 begins may be generalized or may be variable and may depend on the type or types of algae that are selected to be grown. For some embodiments and some modes of operation, the algae may be commercially viable for harvesting at the end of the lipid phase experienced in pond section 128.

The design of pond 104 may be characterized by a slope, length, width, flow rate of water supply, or another parameter to cause algae to proceed through the growth phase and the lipid phase during the operation of pond 104. The result is the production of algae that is commercially viable for harvesting to produce biofuel after a single pass (flow) through pond 104. A slope, length, or width for pond 104 may be defined as an average value for the entire serpentine trough. The slope, length, or width for pond 104 may be defined by selecting individual values for one or more portions of the length of the trough, for example, selected for one or more channel segments 120, one or more of the channel bends 122, or one or more of the pond sections 126, 128.

For example, pond 104 may include an appropriate or sufficient slope, length, slurry depth, or width to retain algae for a residence time within a range of 2 to 25 days (or longer). Unless stated otherwise for specific examples, residence time refers to the time required for algae to make a single pass through pond 104 from inlet 124 to outlet 125. In some examples, a residence time for operation may range between about 4 to 12 days, 8 to 20 days, or 7 to 16 days, as examples. Smaller portions of these ranges of residence time are also contemplated as target values for the design or operation of pond 104. In some embodiments of system 100, the physical design or a selected mode of operation may result in shorter or longer residence times. In some examples, residence time in pond section 126 ranges between about 1 to 5 days, and the residence time in pond section 128 ranges between about 1 to 20 days. Other divisions of residence time among pond sections 126, 128 are possible, without departing from the scope of the disclosure.

In some embodiments, a slope or residence time in pond 104 may be based on the inclusion of one or more weirs (not shown), which may be placed between a channel segment 120 and the subsequent, downhill channel bend 122, between a channel bend 122 and a subsequent, downhill channel segment 120, along the length of a channel segment 120, within a channel bend 122, at one or more other locations along pond 104, or at any combination of these locations. In some embodiments, the height of the weirs may be adjustable to adjust the water level or slurry depth upstream of the given weir. The speed or velocity of algae slurry as it travels through pond 104 during operation may be within a range of 0.1 to 0.6 meters/sec (“m/s”) (0.030 to 0.183 ft/s); although, some ponds 104 may operate at higher or lower velocities. Smaller portions of this velocity range are also contemplated as target values for the design or operation of pond 104.

Referring still to FIG. 1, filtration and pumping system 105 includes a one or more separation devices 106, one or more pumps 108, and a water source or water inlet 110. To be more easily distinguishable, individual devices may be labeled with the corresponding reference numeral and a designating letter, such as A, B, or C. This example includes two separation devices, which can include a skimming device 106A and a membrane filtration unit 106B (e.g., an ultrafiltration membrane unit). Separation devices 106A, 106B are fluidically coupled, directly or indirectly, to receive algae slurry from pond 104 and to separate algae (alternately referred to as “algae biomass”) from the algae slurry. Various types, quantities, and arrangements of separation devices are possible within the scope of this disclosure. As examples, some embodiments may include only skimming device 106A or only membrane filtration unit 106B, and some other embodiments may include multiples of either or both of these units.

A first pump 108A may provide water from water inlet 110 to the inlet 124 of pond 104 via a fluid conduit 114. In some embodiments, the water from water inlet 110 is saline and may be sourced from a body of salt water.

Skimming device 106A is configured and arranged to remove (e.g., to harvest) algae from the algae slurry of pond 104. For this purpose, skimming device 106A is coupled to outlet 125 of pond 104 to receive the algae slurry, and is fluidically coupled to a production conduit 132A to discharge algae, which may be contained in a more highly concentrated algae slurry Skimming device 106A is fluidically coupled to a fluid recycling conduit 134 through a second pump 108B to deliver a remainder of the algae slurry, including water, back to pond 104, to a storage location, to another pond, or to a combination of these. In the present example, recycling conduit 134 is fluidically coupled to conduit 114 and pond inlet 124. A storage or surge tank may be included there between.

Membrane filtration unit 106B is fluidically coupled to pond 104 in series with skimming device 106A, via production conduit 132A to receive the concentrated algae slurry from skimming device 106A and to remove (e.g., to harvest) algae from the algae slurry of pond 104. Filtration unit 106B is fluidically coupled to a production conduit 132B and to a permeate conduit 138. Through production conduit 132B, filtration unit 106B may discharge algae in a more highly concentrated algae slurry and convey the algae to a downstream location 140 for further processing into biofuel or another product. With this arrangement and in this manner, algae may be harvested by skimming device 106A and membrane filtration unit 106B. The harvested algae may be highly separated from water or may be part of an algae slurry that is more highly concentrated than is contained in pond 104.

Permeate conduit 138 conveys a permeate from filtration unit 106B to pump 108B and to recycling conduit 134 in order to deliver the permeate back to pond inlet 124. Thus, for at least some portion of the fluid during some operations, permeate conduit 138 operates as a recycling conduit. Permeate conduit 138 is also fluidically coupled to a purge conduit 142 (alternately referred to as “blowdown”) to discharge steadily or selectively a portion of the permeate for removal from system 100. Discharging a portion of the permeate through purge conduit 142 and receiving new water from water inlet 110 may be performed to maintain, balance, or reduce the overall salinity, pH, or cleanliness of the algae slurry in system 100. In some embodiments purge conduit 142 is also fluidically coupled to skimming device 106A to take fluid from that separation device as well. The flow rates in purge conduit 142 and from water inlet 110, a ratio of these flow rates, or the salinity in pond 104 may be adjustable and, in some embodiments, one or more of these parameters may be monitored or governed by a controller unit that includes suitable computer executable instructions stored in a computer readable medium. In some embodiments, membrane filtration unit 106B may be coupled to receive algae slurry directly from pond 104. System 100 may also include a control unit to monitor and control various aspects of the performance of system 100.

For some embodiments, a majority or all the components of filtration and pumping system 105, or at least pumps 108A,B and separation devices 106A,B and associated plumbing or channels, may be located in a centralized or consolidated location, which may reduce the cost of installing system 100.

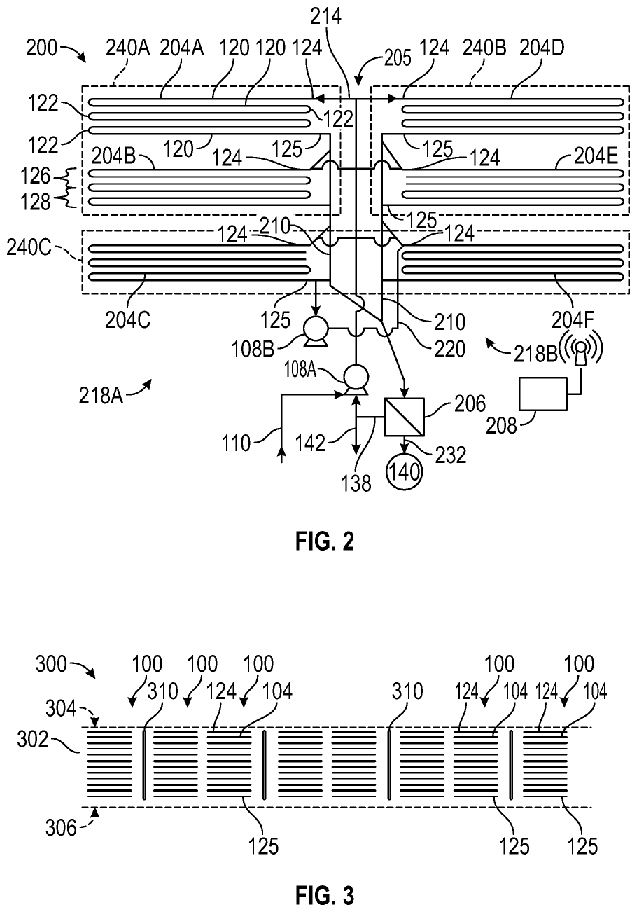

FIG. 2 is a schematic diagram of another example system 200 for growing algae in an algae slurry, according to one or more additional embodiments. System 200 includes a plurality of ponds, shown as ponds 204A, 204B, 204C, 204D, 204E, and 204F. While six ponds 204A-F are depicted in FIG. 2, more or less than six may be included in various embodiments of system 200. The configurable arrangement of system 200 may help compensate for seasonal variations that might otherwise influence algae growth or production rates.

As illustrated, the ponds 204A-F are interconnected by a filtration and pumping system 205, and monitored and controlled by a control unit 208. In at least some embodiments, system 200 may perform as a once-through, gravity-driven system, with respect to the travel of algae though any of ponds 204A-F. Though not shown, an algae seed feed system may be included in the system 200 to provide a continuous or periodic input of algae for growth, which will facilitate a constant algae stream at the exit of each pond 204A-F.

The ponds 204A-F are each formed or shaped as an elongate, serpentine trough. Each pond 204A-F may be gravity-driven and may be built on sloping or graded region of land. The ponds 204A-F may be similar in some respects to pond 104 of FIG. 1. In general, unless specifically described as being different, the configurations and the operations, including the potential variations and terrestrial integration, described for pond 104, are applicable to each of ponds 204A-F. For example, each pond 204A-F includes a plurality of elongate channel segments 120 coupled in series by a plurality of channel bends 122 extending from a corresponding inlet 124 to a corresponding outlet 125, forming the elongate, serpentine flow path for algae slurry. Outlet 125 is located at a lower elevation than inlet 124. The number of channel segments 120 and channel bends 122 shown in FIG. 2 are for illustration purposes only. The sequentially coupled channel segments 120 may be arranged substantially parallel to each other or, alternatively, may be “wrapped around” a hill, as examples. Ponds 204A-F may be exposed to the open air to receive sunlight. Alternatively or in addition, artificial light may be used to help facilitate photosynthesis growth of the algae.

As illustrated, the ponds 204A-F are arranged in a rectangular grid pattern, interconnected by filtration and pumping system 205. In this example, system 200 includes a left-hand group of ponds 204A,B,C, which are listed from highest to lowest elevation, and a right-hand grouping of ponds 204D,E,F, which are listed from highest to lowest elevation. Wherein, the terms left-hand and right-hand refer to the particular orientation and viewpoint shown in the figure, for convenience. In general, ponds 204A and 204D may be horizontally adjacent and located at a similar or different elevation; ponds 204B and 204E may be horizontally adjacent and located at a similar or different elevation; and ponds 204C and 204E may be horizontally adjacent and located at a similar or different elevation.

Filtration and pumping system 205 includes one or more separation devices 206, one or more pumps 108, a configurable fluid transfer conduit 210, a fluid conduit 214, and a water inlet 110 to be coupled to a water source. Fluid transfer conduit 210 interconnects the ponds 204A-F and separation device 206. A pump 108A may provide water from water inlet 110 to one or more ponds 204 via conduit 214. Conduit 214 includes valves or baffles (not shown) and supply branches leading to the inlet 124 of each pond 204. In some embodiments, the water from water inlet 110 is saline and may be sourced from a body of salt water. In at least some embodiments, a majority or all the components of filtration and pumping system 205 are consolidated within a corridor of system 200. For example, in some embodiments, fluid transfer conduit 210, conduit 214, pumps 108A,B, and separation unit 206 are consolidated, located in a central corridor of system 200, located generally between the left-hand group 218A of ponds 204A,B,C and the right-hand group 218B of ponds 204D,E,F. Electrical lines may also be placed primarily or exclusively within the corridor. These placements of equipment in a consolidated or centralized location may reduce the cost of installing system 200 and provide access for repairs and upgrades. Even so, other placements of plumbing lines and electrical lines may be used in some embodiments.

The separation device 206 FIG. 2 may include a membrane filtration unit similar in some respects to membrane filtration unit 106B of FIG. 1. In general, unless specifically described as being different, the configuration and operation, including the potential variations, described for filtration unit 106B are applicable to separation device 206. Separation device 206 may further or alternatively include a skimming device, with or without a membrane filtration unit. The skimming device may be similar to skimming device 106A of FIG. 1, for example. Separation device 206 is fluidically coupled, to one or more of the ponds 204A-F via fluid transfer conduit 210 to receive algae slurry. Separation device 206 may separate algae from a remainder of the slurry; i.e., “permeate”. The permeate may include water, nutrients, and, possibly, residual algae. Separation device 206 is also fluidically coupled to a production conduit 232 and to a permeate conduit 138. Through production conduit 232, separation device 206 may discharge algae, which may comprise a highly concentrated algae slurry, and may provide this product to downstream location 140 for further processing into biofuel or another product.

Permeate conduit 138 is coupled to pump 108A or another pump to deliver the permeate back to one or more of the ponds 204A-F via conduit 214. Permeate conduit 138 is also fluidically coupled to a purge conduit 142 to discharge steadily or selectively a portion of the permeate in order to maintain, balance, or reduce the overall salinity, pH, or cleanliness of the slurry in system 200. Thus, for at least some portion of the fluid during some operations, permeate conduit 138 performs as a recycling conduit. Alternately, some or all the permeate may be delivered to a storage location.

In some modes of operations, a second pump 108B and a corresponding fluid by-pass conduit 220 fluidically couple the outlet 125 of pond 204C with the inlet 124 of an adjacent pond 204F. Transfer conduit 210 is configurable to deliver algae slurry to separation device 206 for harvesting and to deliver algae slurry from one pond 204A-F to another pond 204A-F to increase a residence time and achieve further growth of the algae, depending on the selected configuration of valves or baffles included in transfer conduit 210, as described below.

The salinity, pH, or cleanliness of ponds 204 and the destination of the permeate of system 200 may be governed by a controller unit 208. More specifically, various aspects of the operation of system 200 may be monitored or controlled by control unit 208 or may be manually controlled using instrumentation, manual valves, baffles, or weir level adjustments, as examples. In various embodiments, control unit 208 may operate in an automated mode, a remote control mode operated by a user, or a combination of these modes. Various embodiments of system 200 are configurable for multiple modes of operation. The various valves (not shown) in supply conduit 214 and valves, baffles, or weirs, etc. (not shown) in transfer conduit 210 may be open, closed, raised, lowered, or modulated to direct the flow of water, nutrients, and algae slurry in order to select a particular mode of operation for system 200. Other adjustments to system 200 may also be performed when selecting a mode of operation. In an example, system 200 is configurable for three modes of operation. For a first mode of operation, each pond 204 operates individually, operating in parallel with the other ponds 204. In the first mode, each pond 204A-F may be fluidically coupled to pump 108A and conduit 214 to receive new water or permeate, and each pond 204 may be fluidically coupled to transfer conduit 210 to deliver algae product to separation device 206 for harvesting.

For a second mode of operation, system 200 is divided into groups of ponds 204A-F by appropriate adjustments made to the valves, baffles, or weirs mentioned above, to transport slurry sequentially through the ponds of the group. FIG. 2 shows three groups 240A, 240B, 240C of ponds 204A-F, which in this example are sequential or cascaded pairs indicated by dashed boxes. The ponds within each group 240A,B,C are configured to operate in series, and the groups 240A,B,C are configured to operate in parallel. The first group 240A includes ponds 204A,B, a second group 240B includes ponds 204D,E, and a third group 240C includes ponds 204C,F.

For ponds 204A,B to operate in series, pond 204A is fluidically coupled to receive fluid from pump 108A and conduit 214, and the outlet 125 of pond 204A is fluidically coupled to the inlet 124 of pond 204B through a portion of transfer conduit 210, which is isolated from the remainder of transfer conduit 210. Also, pond 204B is fluidically coupled by another portion of transfer conduit 210 to deliver algae product to separation device 206. This arrangement extends the length of the flow path in which algae may grow, increasing residence time during operation in at least some modes of operation. Group 240A is configured so that pond 204B receives slurry from pond 204A, and the inlet 124 of pond 204B is isolated from direct fluid communication with pump 108A and conduit 214. Also, pond 204A is isolated from direct fluid communication with separation device 206.

The second group 240B includes ponds 204D,E fluidically coupled similar to the arrangement of ponds 204A,B of group 240A. In at least some embodiments, gravity drives a flow of algae slurry through pond 204A and pond 204B, and gravity drives a flow of algae slurry through pond 204D and pond 204E.

The third group 240C includes ponds 204C,F fluidically coupled similar to the arrangement of ponds 204A,B of group 240A with the addition that second pump 108B and by-pass conduit 220 fluidically couple the outlet 125 of pond 204C with the inlet 124 of pond 204F. During the second mode of operation pump 108B lifts algae slurry against gravity, passing the algae from pond 240C to pond 240F, which may be located a similar elevations. In some arrangements of system 200, pond 204F is located at a lower elevation than pond 204C, and consequently, pump 108B is not included, or pump 108B may not be operated during the second mode of operation. In the second mode of operation, groups 240A,B,C operate in parallel.

For a third mode of operation, system 200 is also divided into groups of 204A-F by appropriate adjustments made to the valves, baffles, or weirs of system 200. The groups of the third mode include more ponds than are included in the groups of the second mode. In FIG. 2, as defined above, the left-hand group 218A includes ponds 204A,B,C configured to operate in series, and the right-hand group 218B includes ponds 204D,E,F configured to operate in series, to extend the length of the flow path in which algae may grow. Thus, each group 218A,B includes three sequential or cascaded ponds in this example. The groups 218A,B are configured to operate in parallel. For the third mode of operation, pond 204A is fluidically coupled to pump 108A and conduit 214. The outlet 125 of pond 204A is fluidically coupled to the inlet 124 of pond 204B through a portion of transfer conduit 210, and the outlet 125 of pond 204B is fluidically coupled to the inlet 124 of pond 204C through another portion of transfer conduit 210. Pond 204C is fluidically coupled to transfer conduit 210 to deliver algae product to separation device 206. The inlets 124 of ponds 204B,C are isolated from direct fluid communication with pump 108A and conduit 214. The outlets 125 of ponds 204A,B are isolated from direct fluid communication with separation device 206.

Ponds 204D,E,F of the right-hand group 218B are fluidically coupled similar to the arrangement of ponds 204A,B,C of group 218A. In at least some embodiments, gravity drives a flow of algae slurry through ponds 204A,B,C, and gravity drives a flow of algae slurry through ponds 204D,E,F. In some embodiments, by-pass conduit 220 may be adjusted to fluidically couple the outlet 125 of pond 204C with the inlet 124 of pond 204D to allow all six ponds 204 to operate in series. Some embodiments of system 200 may include more than six ponds 204 and may include more the two or three group of ponds operating in series or may include groups of four, five, six, or more ponds 204 operating in series.

The ability to be reconfigured for multiple modes of operation, may allow system 200 to compensate for seasonally varying growth rates of algae and may cause algae of a desired maturity to be received at separation device 206 during various seasons of the year. Configuring and operating system 200 for the first, second, or third modes of operation can modify the residence time of algae slurry within the various ponds and groups of ponds in system 200. The first mode of operation, in which the shortest flow path is utilized, may be useful for summer conditions, in which algae may grow the quickest. The second mode of operation, having a longer flow path, may be useful for fall and spring season, when algae may grow slower than during summer. The third mode of operation, having a still longer flow path for each group 218A, 218B, may be useful for the winter season when algae may grow the slowest. As will be appreciated, the appropriateness of these seasonal adjustments may be dependent on the particular location chosen for a system 200. Systems having more than six ponds may provide even more adjustment to the residence time of algae slurry. Some embodiments may be configurable for only two modes or for more than three modes of operations, which may depend on the number of ponds that are available to be coupled fluidically in series, for example. In some examples, the number of ponds in a group of ponds operating in series is a value in the range of two to five or in the range of two to ten. Some embodiments, may operate with more than ten ponds fluidically coupled in series.

Changing between some modes of operation for system 200 includes changing the length of the flow path through which the algae and its slurry travel prior to being delivered to separation device 206 for harvesting. In general, a longer flow path corresponds to a longer residence time for growth of the algae and vice versa. Depending on the change needed, the flow path may be lengthened or shortened. For embodiments in which each pond 204 (e.g., each separate flow path) has the same or a similar length, the increase in flow path length when changing from the first mode to the second mode is substantially 100%, the increase in flow path length when changing from the first mode directly to the third mode is substantially 200%, the increase in flow path length when changing from the second mode to the third mode is substantially 50%. The decreases in length of flow path when making the opposite changes in mode of operation may be directly determined. As examples, a reduction in flow path length may be in the range of 33% to 68% for the changes between the third, second, and first modes discussed herein. Other increases and decreases in a flow path length are possible depending on the individual lengths of the ponds and the numbers of ponds that may be joined in series or separated.

In various embodiments and various modes of operation, the ranges of residence times described for system 100 of FIG. 1 also apply to system 200 of FIG. 2. Depending on factors such as the season of the year, the selected mode of operation, type of algae, and provision of nutrients, as examples, the residence times and ranges of residence times described for pond 104 of system 100 may pertain individually to one or more of the ponds 204A-F, or may pertain individually to one or more groups 240A, 240B, 240C of ponds 204, or may pertain individually to one or more groups 218A,B of ponds 204. Unless stated otherwise for specific examples, residence times refer to the time that transpires while algae makes a single pass through a pond or through a group of ponds coupled in series. In some examples, the residence time for one of the ponds 204A-F is 4.5 days. Based on the same or related factors, pond sections 126, 128 may be defined or identified for individual ponds 204, for individual groups 240A, 240B, 240C, or for individual groups 218A,B. Thus, any of these ponds or pond groups may include a first pond section 126 in which algae may proceed through a growth phase and a second pond section 128 in which algae may proceed through a lipid phase, as previously described. FIG. 2 shows a pair of pond sections 126, 128 associated with pond 204B, as an example.

FIG. 3 is a schematic diagram of an example system 300 for growing algae, which may incorporate multiples of the system 100 of FIG. 1 or system 200 of FIG. 2, in various combinations, according to one or more embodiments. For simplicity, system 300 will be described as including multiples of the system 100 of FIG. 1. System 300 may be situated on a region of land 302 having a downward slope from an upper land portion 304 to a lower land portion 306. The elevation across upper land portion 304 or across lower land portion 306 may vary. A plurality of systems 100 are located on land 302. Each system 100 including a pond 104 formed or shaped as an elongate, serpentine trough, as previously described, extending from an inlet 124 at the upper portion 304 to an outlet 125 at the lower land portion 306. Each system 100 further includes a filtration and pumping system 105 (FIG. 1), as previously described. Alternatively, one or more systems 100 may share all or portions of one or more filtration and pumping system 105 to utilize more effectively the available land area or to reduce capital costs, operating costs, or maintenance costs. Roadways 310 for maintenance or other purposes are shown between various, neighboring systems 100.

Some embodiments of systems 100, 200, 300, are configured or operated to vary the slurry depth for a pond 104, 204A-F dependent on the time of year (season). Changing the slurry depth may involve raising or lowering the height of weirs, changing the feed rate of water to the inlet 124 of the pond 104, or changing the rate of recycling slurry through permeate conduit 138 and pump 108A. Relative to a mean or a minimum slurry depth for the pond, as examples, increasing the slurry depth in the pond is anticipated to increase the flow velocity of algae slurry and to reduce residence time, which may be appropriate during seasons or time of year when algae has a relatively high growth rate. As a result, the production rate of algae may be increased during these seasons as compare to a pond that used a lower depth during the same season. Increasing the slurry depth may be advantageous during the summer season or during all or portions of the spring or fall seasons because of the increased amount of solar interaction.

Alternatively, using a lower slurry depth in the pond is anticipated to decrease the flow velocity of algae slurry within the pond and to increase residence time, which may be appropriate during seasons or time of year when algae has a relatively low growth rate. As a result, a desired production effectiveness of the pond may be achieved or maintained despite the lower growth rate that the algae may experience during such times. The reduced depth may allow deeper penetration of the solar rays into the slurry, which helps the algae proceed more completely through the growth phase or the lipid phase to produce algae of a desired maturity level for harvesting. Using decreased slurry depth and velocity may be advantageous during the winter season or during all or portions of the spring or fall seasons. In some embodiments, the slurry depth and velocity may be varied based on a season of a calendar year corresponding to calendar months extending from a month with hottest temperatures on average (e.g., July or August in North America) to a month with coldest temperatures on average (e.g., January or February in North America). In such embodiments, the depth of the slurry may be reduced by about 35% to about 65% during this season, and the velocity of the slurry may be reduced by about 50% to about 75% during the season. Moreover, the slurry depth and velocity of a pond may be varied incrementally as the seasons change. The process of selecting (e.g., maintaining, increasing, or decreasing) the slurry depth and velocity may be combined with the process of operating multiple ponds in series as described with respect to system 200 of FIG. 2 to achieve a targeted residence time for algae slurry.

### EXAMPLE EMBODIMENTS

An example embodiment of a pond 104, 204A-F includes a slope of 0.5% for at least some of the channels 120. The existing slope of a land region on which the pond is built may be more, less, or generally equal to the targeted slope of 0.5%, and excavation or built of the land region may be performed to achieve the targeted slope. The example embodiment includes eight channel segments 120 and seven channel bends 122. Each channel segment 120 has a length of 3.3 km (2.0 mile), yielding a flow path length of 26 km (16 miles), not accounting for the channel bends 122. The dimensions and planned operating conditions of channel segments 120 include a width of 31.55 m (130.5 ft), a slurry depth of 0.254 m (0.833 ft; 10 inches), a slurry velocity at 0.3 m/s (0.09 ft/s), and a Manning's “n” value equal to 0.012. Example embodiments include ponds 104, 204A-F having a slurry depth ranging from 0.152 to 0.318 m (0.500 to 1.042 ft; 6 to 12.5 inches) and a slurry velocities ranging from 0.25 m/s to 0.40 m/s (0.82-1.3 ft/s).

The present disclosure provides, among others, the following embodiments, each of which may be considered as alternatively including any of the alternate embodiments.

Clause 1. A system for growing algae in a slurry that includes a pond having an inlet and an outlet and a plurality of contiguous elongate channel segments coupled fluidically in sequence between the inlet and the outlet, and the inlet is located at an elevation higher than outlet to allow gravity to flow the slurry from the inlet to the outlet. The system further includes a separation device fluidically coupled to the outlet to receive and separate algae from the slurry.

Clause 2. The system of Clause 1, wherein the separation device comprises a membrane filtration unit and the slurry is recycled back to the inlet from the membrane filtration unit after separating the algae.

Clause 3. The system of Clause 2, wherein the separation device further comprises a skimming device fluidically coupled to the membrane filtration unit and interposing the outlet and the membrane filtration unit.

Clause 4. The system of any of the Clauses 1 to 3, wherein a residence time of the algae slurry to flow between the inlet and the outlet ranges between about 4 days and about 25 days.

Clause 5. The system of any of the Clauses 1 to 4, wherein the pond is a first pond and the system further comprises a second pond having an inlet and an outlet and a plurality of contiguous elongate channel segments coupled fluidically in sequence between the inlet and the outlet of the second pond; wherein, the inlet of the second pond is located at an elevation higher than outlet of the second pond to allow gravity to flow the slurry from the inlet of the second pond to the outlet of the second pond; and wherein the separation device is fluidically coupled to the outlet of the second pond to receive and separate algae from the slurry within the second pond.

Clause 6. The system of Clause 5, further comprising a fluid transfer conduit interconnecting the first and second ponds and the separation device, the fluid transfer conduit being configurable for a first mode of operation where the first and second ponds are fluidically coupled to operate in parallel, and the fluid transfer conduit is fluidically coupled to transfer slurry from each pond to the separation device.

Clause 7. The system of Clause 6, wherein the fluid transfer conduit is configurable for a second mode of operation where the first and second ponds are fluidically coupled to operate in series, and the fluid transfer conduit is fluidically coupled to transfer the slurry from the first pond to the second pond and transfer the slurry from the second pond to the separation device.

Clause 8. The system of Clause 7, wherein the fluid transfer conduit is configurable for a third mode of operation in which the fluid transfer conduit is fluidically coupled to transfer slurry from the first pond to the second pond and from the second pond to a third pond having an inlet and an outlet and a plurality of contiguous elongate channel segments coupled fluidically in sequence between the inlet and the outlet of the third pond, and wherein the fluid transfer conduit is fluidically coupled to transfer slurry from the third pond to the separation device.

Clause 9. The system of any of the Clauses 1 to 8, wherein the pond is a member of a plurality of ponds, each pond including an inlet and an outlet and a plurality of contiguous elongate channel segments coupled fluidically in sequence between the inlet and the outlet, wherein the inlet is located at an elevation higher than outlet to allow gravity to flow the slurry from the inlet to the outlet; wherein, the system further comprises a fluid transfer conduit interconnecting the plurality of ponds and the separation device, the fluid transfer conduit being configurable for a first mode of operation where of the ponds of the plurality are fluidically coupled to operate in parallel with the fluid transfer conduit being fluidically coupled to transfer the slurry from each pond to the separation device.

Clause 10. The system of any of the Clauses 1 to 9, wherein the fluid transfer conduit is configurable for a second mode of operation where the fluid transfer conduit fluidically couples a first group of the ponds to operate in series to transport the slurry sequentially through each pond of the first group of ponds and to deliver the slurry to the separation device.

Clause 11. The system of Clause 10, wherein for the second mode of operation, the fluid transfer conduit configures a second group of the ponds to operate in parallel with respect to the first group of the ponds.

Clause 12. The system of Clause 11, wherein for the second mode of operation, the fluid transfer conduit fluidically couples the second group of the ponds to operate in series to transport the slurry through each pond of the second group of the ponds and to deliver the slurry to the separation device.

Clause 13. The system of Clause 12, wherein a recycling conduit is fluidically coupled between the separation device and the plurality of ponds; wherein for the first mode of operation, the recycling conduit is configured to deliver portions of the first remainder of the slurry to each pond, and wherein for the second mode of operation the recycling conduit is configured to deliver portions of the first remainder of the slurry to the first and second groups of the ponds.

Clause 14. A method for growing algae includes containing a slurry that includes algae in a pond having an inlet and an outlet and a plurality of contiguous elongate channel segments coupled fluidically in sequence between the inlet and the outlet, wherein the inlet is located at an elevation higher than outlet, flowing the slurry between the inlet and the outlet under force of gravity, receiving the slurry from the pond at a separation device, and separating the slurry into algae and a remainder of the slurry with the separation device.

Clause 15. The method of Clause 14 further comprising: transferring a first portion of the remainder of the slurry to the pond, and disposing of a second portion of the remainder of the slurry.

Clause 16. The method of any of the Clauses 14 to 15 wherein the pond comprises a plurality of ponds, each pond including an inlet and an outlet and a plurality of contiguous elongate channel segments coupled fluidically in sequence between the inlet and the outlet; wherein, the method further comprises: performing a first mode of operation, which includes conveying portions of the slurry through each of ponds in parallel and transferring the slurry from each pond to the separation device, and performing a second mode of operation, which includes conveying the slurry sequentially through each pond of a first group of the ponds and transferring the slurry from a final pond of the first group to the separation device.

Clause 17. The method of Clause 16, wherein the second mode of operation further comprises: operating a second group of the ponds in parallel with respect to the first group; and conveying a portion of the slurry sequentially through each pond of the second group and transferring the slurry from a final pond of the second group to the separation device.

Clause 18. The method of any of the Clauses 16 to 17, further comprising: conveying a portion of the remainder of the slurry to each pond during the first mode of operation; and conveying a portion of the remainder of the slurry to one pond of the first group during the second mode of operation.

Clause 19. The method of any of the Clauses 16 to 20, wherein while performing the second mode of operation, the first group of the ponds is characterized by a flow path length, along which the algae travels, that is in the range of 50% to 200% greater than a flow path length of at least one of the ponds of first group while performing the first mode of operation.

Clause 20. The method of ay of Clauses 14 to 19, further comprising altering at least one of a depth and a velocity of the slurry based on season.

Clause 21. The method of Clause 20, wherein the season comprises calendar months extending from a month with hottest temperatures on average to a month with coldest temperatures on average, the method further comprising reducing the depth of the slurry by about 35% to about 65% during the season, and reducing the velocity of the slurry by about 50% to about 75% during the season.

### Nomenclature

The terms used herein, including the claims, have their plain, ordinary meaning unless otherwise explicitly and clearly defined by the patentee. The indefinite articles “a” or “an,” as used herein, including the claims, are defined herein to mean one or more than one of the element that it introduces. The term “or” as used in a phrase such as “A or B” herein is intended to include alternatively of any of the following: “A” alone, “B” alone, and, where feasible, “A and B.” Ordinal numbers such as first, second, third, etc. do not indicate a quantity but are used for naming and reference purposes. In addition, ordinal numbers used in the claims in reference to a component or feature may differ from the ordinal numbers used in the written description for the corresponding component or feature. For example, a “second object” in a claim might be described as a “third object” or may be described without an ordinal number in the written description.

As used herein, including the claims, a “line” for fluid communication may include any of the following pipe, piping, tubing, hose, fittings, valves, gauges, check valves, flow meters, filters, closed channel members, and the like. In some embodiments, a portion or the entirety of one or more pipe or “line” may be replaced by open or closed channels or troughs when suitable, e.g., when a portion or entirety of the flow path for the pipe or line extends downward in the intended direction of flow. As used herein, including the claims, the terms “trough” and “channel” are used interchangeably to refer to an open channel. In some embodiments, a portion or the entirety of some channels or troughs may be replaced by line for fluid communication or may be covered. For some embodiments, on or more troughs or channels may be formed by digging, carving, or building a trench in or on an earthen formation. As used herein, including the claims, a “conduit” for fluid communication, may include any of piping, a fluid communication line, an open or closed channel, a trough, or any combination of these.

Unless otherwise indicated, all numbers expressing quantities of ingredients, properties such as length, volume, mass, molecular weight, operating conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a to b”) disclosed herein is to be understood to set forth every number and range encompassed within the broader range of values.

For the sake of clarity, not all features of a physical embodiment are described or shown in this application. It is understood that in the development of a physical embodiment incorporating the embodiments of the present disclosure, numerous implementation-specific decisions must be made to achieve the developer's goals, such as compliance with system-related, business-related, government-related, and other constraints, which vary by implementation and from time to time. While a developer's efforts might be time-consuming, such efforts would be, nevertheless, a routine undertaking for those of ordinary skill in the art and having benefit of this disclosure.

Therefore, the disclosed apparatuses, systems, and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present invention. The apparatuses, systems, and methods illustratively disclosed herein suitably may be practiced in the absence of any element that is not specifically disclosed herein and/or any alternative element disclosed herein. While components, compositions, and methods are described in terms of “comprising,” “containing,” or “including” various components or steps, the components, compositions, and methods can also “consist essentially of” or “consist of” the various components and steps. For the methods herein, the order of various process steps may be rearranged in various embodiments and yet remain within the scope of the disclosure, including the claims.

The invention claimed is:

1. A system for growing algae in a slurry, comprising:
a pond having an inlet and an outlet and a plurality of contiguous elongate channel segments coupled fluidically in sequence between the inlet and the outlet, wherein the inlet is located at an elevation higher than the outlet to allow gravity to flow the slurry from the inlet to the outlet; and
a separation device fluidically coupled to the outlet to receive and separate algae from the slurry.

2. The system of claim 1, wherein the separation device comprises a membrane filtration unit and the slurry is recycled back to the inlet from the membrane filtration unit after separating the algae.

3. The system of claim 2, wherein the separation device further comprises a skimming device fluidically coupled to the membrane filtration unit and interposing the outlet and the membrane filtration unit.

4. The system of claim 1, wherein a residence time of the algae slurry to flow between the inlet and the outlet ranges between about 4 days and about 25 days.

5. The system of claim 1, wherein the pond is a first pond and the system further comprises a second pond having an inlet and an outlet and a plurality of contiguous elongate channel segments coupled fluidically in sequence between the inlet and the outlet of the second pond,
wherein the inlet of the second pond is located at an elevation higher than outlet of the second pond to allow gravity to flow the slurry from the inlet of the second pond to the outlet of the second pond, and
wherein the separation device is fluidically coupled to the outlet of the second pond to receive and separate algae from the slurry within the second pond.

6. The system of claim 5, further comprising a fluid transfer conduit interconnecting the first and second ponds and the separation device, the fluid transfer conduit being configurable for a first mode of operation where the first and second ponds are fluidically coupled to operate in parallel, and the fluid transfer conduit is fluidically coupled to transfer slurry from each pond to the separation device.

7. The system of claim 6, wherein the fluid transfer conduit is configurable for a second mode of operation where the first and second ponds are fluidically coupled to operate in series, and the fluid transfer conduit is fluidically coupled to transfer the slurry from the first pond to the second pond and transfer the slurry from the second pond to the separation device.

8. The system of claim 7, wherein the fluid transfer conduit is configurable for a third mode of operation in which the fluid transfer conduit is fluidically coupled to transfer slurry from the first pond to the second pond and from the second pond to a third pond having an inlet and an outlet and a plurality of contiguous elongate channel segments coupled fluidically in sequence between the inlet and the outlet of the third pond, and wherein the fluid transfer conduit is fluidically coupled to transfer slurry from the third pond to the separation device.

9. The system of claim 1, wherein the pond is a member of a plurality of ponds, each pond including an inlet and an outlet and a plurality of contiguous elongate channel segments coupled fluidically in sequence between the inlet and the outlet, wherein the inlet is located at an elevation higher than outlet to allow gravity to flow the slurry from the inlet to the outlet,
wherein the system further comprises a fluid transfer conduit interconnecting the plurality of ponds and the separation device, the fluid transfer conduit being configurable for a first mode of operation where of the ponds of the plurality are fluidically coupled to operate in parallel with the fluid transfer conduit being fluidically coupled to transfer the slurry from each pond to the separation device.

10. The system of claim 9, wherein the fluid transfer conduit is configurable for a second mode of operation where the fluid transfer conduit fluidically couples a first group of the ponds to operate in series to transport the slurry sequentially through each pond of the first group of ponds and to deliver the slurry to the separation device.

11. The system of claim 10, wherein for the second mode of operation, the fluid transfer conduit configures a second group of the ponds to operate in parallel with respect to the first group of the ponds.

12. The system of claim 11, wherein for the second mode of operation, the fluid transfer conduit fluidically couples the second group of the ponds to operate in series to transport the slurry through each pond of the second group of the ponds and to deliver the slurry to the separation device.

13. The system of claim 12, wherein a recycling conduit is fluidically coupled between the separation device and the plurality of ponds,
wherein for the first mode of operation, the recycling conduit is configured to deliver portions of the first remainder of the slurry to each pond, and
wherein for the second mode of operation the recycling conduit is configured to deliver portions of the first remainder of the slurry to the first and second groups of the ponds.

14. A method for growing algae, comprising:
containing a slurry that includes algae in a pond having an inlet and an outlet and a plurality of contiguous elongate channel segments coupled fluidically in sequence between the inlet and the outlet, wherein the inlet is located at an elevation higher than outlet;
flowing the slurry between the inlet and the outlet under force of gravity;
receiving the slurry from the pond at a separation device; and
separating the slurry into algae and a remainder of the slurry with the separation device.

15. The method of claim 14, further comprising:
transferring a first portion of the remainder of the slurry to the pond; and
disposing of a second portion of the remainder of the slurry.

16. The method of claim 14, wherein the pond comprises a plurality of ponds, each pond including an inlet and an outlet and a plurality of contiguous elongate channel segments coupled fluidically in sequence between the inlet and the outlet, method further comprising:
performing a first mode of operation, which includes conveying portions of the slurry through each of ponds in parallel and transferring the slurry from each pond to the separation device; and
performing a second mode of operation, which includes conveying the slurry sequentially through each pond of a first group of the ponds and transferring the slurry from a final pond of the first group to the separation device.

17. The method of claim 16, wherein the second mode of operation further comprises:
operating a second group of the ponds in parallel with respect to the first group; and
conveying a portion of the slurry sequentially through each pond of the second group and transferring the slurry from a final pond of the second group to the separation device.

18. The method of claim 16, further comprising:
conveying a portion of the remainder of the slurry to each pond during the first mode of operation; and
conveying a portion of the remainder of the slurry to one pond of the first group during the second mode of operation.

19. The method of claim 16, wherein while performing the second mode of operation, the first group of the ponds is characterized by a flow path length, along which the algae travels, that is in the range of 50% to 200% greater than a flow path length of at least one of the ponds of first group while performing the first mode of operation.

20. The method of claim 14, further comprising altering at least one of a depth and a velocity of the slurry based on season.

21. The method of claim 20, wherein the season comprises calendar months extending from a month with hottest temperatures on average to a month with coldest temperatures on average, the method further comprising:
reducing the depth of the slurry by about 35% to about 65% during the season; and
reducing the velocity of the slurry by about 50% to about 75% during the season.
